(19) United States
(12) Patent Application Publication
(10) Pub. No.: US 2025/0152881 A1
(43) Pub. Date: May 15, 2025

(54) Shield for a tracheostomy cannula

(71) Applicant: Coloplast A/S, Humlebaek (DK)

(72) Inventor: RALF SCHNELL,, Seligenstadt (DE)

(21) Appl. No.: 18/833,563

(22) PCT Filed: Feb. 1, 2023
(86) PCT No.: PCT/EP2023/052449

§ 371 Date: Jul. 26, 2024

(30) Foreign Application Priority Data
Feb. 4, 2022 (DE) 102022102715.5
Feb. 1, 2023 (WO) PCT/EP2023/052449

(51) Int. Cl. A61M 16/04

(52) U.S. Cl. CPC A61M 16/0465; A61M 2205/0216; A61M 2209/088; A61M 2210/1032

(57) ABSTRACT

To provide a shield for mounting on a cannula tube of a tracheostomy cannula, in which the cannula strap has no skin contact where it is threaded into the eyelet, and with no undesirable lever and tilt effects when applying and fastening the shield, it is proposed according to the invention that the shield has a central region and two side parts extending radially from the central region, wherein the shield has a proximal surface facing away from the patient's neck and a distal surface facing toward the patient's neck, wherein the two side parts each have an eyelet for fastening a retaining strap, wherein the respective eyelet is formed by a bridge-like fastening element which has two pillar portions arranged on the proximal surface of the shield and a bridging portion extending between the two pillar portions, wherein both pillar portions are arranged at a distance from the end of the respective side part.

The present invention relates to a shield for mounting on a cannula tube of a tracheostomy cannula, optionally via an annular connector arranged on the cannula tube, wherein the shield has a central region and two side parts which adjoin two opposite sides of the central region and extend radially from there to the respective end of the side part, wherein the two side parts each have an eyelet through which a retaining strap is guided. In addition, the present invention relates to a tracheostomy cannula for insertion into a tracheostoma, with a cannula tube and with a shield of the type according to the invention arranged on the cannula.

Tracheostomy tubes, which are also often referred to as tracheostomy cannulas, are usually equipped with a neck plate, which is also referred to as a cannula shield, so that the tube or the cannula does not penetrate any deeper than desired into the trachea. Commercially available cannula shields are typically plate-shaped and have an eyelet on each of two sides, on which eyelet the cannula strap can be threaded. The eyelets are typically provided here simply in the form of continuous recesses in the plate-shaped shield.

The cannula strap is required for fixing the cannula to the patient's neck so as to prevent the cannula from accidentally slipping out of the tracheostoma. The cannula strap is guided for these purposes around the patient's neck and is fastened in each case on the eyelets of the cannula shield, which are arranged at the end of the left and right side parts.

The fastening of the cannula strap to the eyelet can be carried out by forming a loop with the strap, wherein the cannula strap is fixed to itself via a velcro-type fastener. Alternatively, hooks for inserting into the eyelet can also be provided at the ends of the cannula straps. Some cannula straps are also simply tied securely onto the eyelet of the cannula shield. Some users also use the eyelets to suture cannula shields to the patient's neck.

Cannula straps that are threaded through eyelets at the end of the side parts of a cannula shield often cause skin irritation. This is due to the fact that the eyelet in many cannula shields is arranged in such a way that a part of the cannula strap runs between the patient's neck and the shield surface facing the patient's neck, thus constantly rubbing against the patient's skin as the patient moves.

U.S. Pat. No. 8,522,788 B2 discloses a cannula shield in which, at the ends of the shield, a gradual thickening is provided in which an eyelet for fastening a retaining strap is then formed. In some embodiments, the eyelet is designed so that the retaining strap can be guided in such a way that it does not run between the shield and the patient's neck.

DE 10 2011 006 064 describes a cannula shield in which the outermost ends of the cannula shield curve away from the patient's neck, and retaining eyelets are let into the shield material in this curved end region in each case. The curvature at the ends of the side parts means that the eyelet does not lie at the height of the patient's neck, and therefore the retaining strap can be guided such that it does not run between the shield and the patient's neck.

The two aforementioned prior art publications only partially solve the problem of skin irritation by guiding the cannula strap through the eyelet slightly above the level of the skin. Especially in obese patients, however, the cannula strap often presses the cannula shield so deep into the soft tissue that the cannula strap still has contact with the skin, and, moreover, the cannula eyelet does not then always lie free enough to allow a new cannula strap to be inserted without lifting the shield slightly. The latter is usually very unpleasant for the patient and leads to coughing or even a choking sensation.

It has also been found that, when the cannula strap is fastened to lateral eyelets above the level of the skin, certain lever and tilt effects may be observed in the case of unilateral loads and in the case of certain patient anatomies, and these effects lead to tilting of the shield, which can be quite unpleasant for the patient. This can be especially seen in particularly slim patients when the cannula strap is loaded on one side.

Against this background, the object of the present invention was to seek a solution by which it can be ensured that the cannula strap has no skin contact where it is threaded into the eyelet, and with no possibility of undesired lever and tilt effects occurring when applying and fastening the shield.

According to the invention, this object is achieved by a shield for mounting on a cannula tube of a tracheostomy cannula or on an annular connector arranged proximally on the cannula tube, wherein the shield has a central region comprising a continuous recess through which the cannula tube and/or the connector is guided, and two side parts which adjoin two opposite sides of the central region and extend radially from there to the respective end of the side part, wherein the shield has a proximal side with a proximal surface, which faces away from the patient's neck during use, and has a distal side with a distal surface, which faces toward the patient's neck during use, wherein the two side parts each have an eyelet for fastening a retaining strap, characterized in that the respective eyelet is formed by a bridge-like fastening element which has two pillar portions arranged on the proximal surface of the shield and a bridging portion which extends between the two pillar portions, wherein both pillar portions are arranged at a distance from the end of the respective side part.

In the context of the present invention, the terms “distal” and “proximal” are used from the perspective of a physician using the ventilation device, i.e. the proximal end is that end of the ventilation device which remains outside the patient's body after insertion into the trachea, while the distal end is inserted into the patient's trachea.

The fact that in the present invention the respective eyelet on the side parts of the shield is formed by a bridge-like fastening element, whose pillar portions are arranged on the proximal surface of the shield at a distance from the end of the respective side part, prevents a situation where part of the cannula strap fastened to the eyelet can become trapped between shield and skin and can thus cause skin irritation.

In addition, when applying the cannula strap and inserting and fastening it, there is in this way much less likelihood of unfavorable lever and tilt effects which can lead to tilting of the shield on the patient's neck. In any case, by virtue of the arrangement proposed according to the invention of a bridge-like fastening element at a distance from the end of the side parts, the tendency to tilt is much lower by comparison with shields in which the eyelets for the retaining strap are arranged at the outermost end of the side parts of the shield.

In certain embodiments, the pillar portions of the bridge-like fastening element stand on the proximal surface of the shield at the same distance from the outermost end of the respective side part. In alternative embodiments, the pillar portions of the bridge-like fastening element are at different distances from the outermost end of the respective side part. The distance from the end of each side part depends, among other things, on how the shield is cut geometrically.

Based on a projection of the central longitudinal axis of the side part on its proximal surface, the pillar portions of a fastening element can stand on a line that runs on the longitudinal axis or parallel to it. Here, the central longitudinal axis denotes the central axis of the shield in the longitudinal direction from the outer end of one side part to the outer end of the other side part. In alternative embodiments, the pillar portions of a fastening element on the proximal surface of the side part stand on a line which intersects the projection of the central longitudinal axis of the side part on its proximal surface at an angle of 45 to 90°.

In some embodiments, the two pillar portions of the bridge-like fastening element arranged on a side part can be arranged mirror-symmetrically with respect to the projection of the central longitudinal axis of the side part on its proximal surface. Depending on the embodiment, it is also possible to deviate from this form of symmetry. In certain embodiments, the two fastening elements arranged on the two side parts of the shield are arranged mirror-symmetrically with respect to a projection of the central transverse axis of the shield.

The two pillar portions of the bridge-like fastening element and the bridging portion extending between them form, together with the proximal surface of the side part, an eyelet through which a retaining strap can be guided and/or to which the neckstrap can be fastened. In certain embodiments, the distance between the inner sides of the pillar portions of a bridge-like fastening element measures 3 to 12 mm. The clear height between the proximal surface of the side part and the inner side of the bridge portion is preferably in the range of 2 to 5 mm. With these dimensions, an eyelet is formed through which most commercially available retaining straps can be easily guided.

To keep the height by which the bridge-like fastening element lies over the proximal surface of the shield as low as possible, in some embodiments a recess is provided in the proximal surface under the bridge-like fastening element. In some embodiments, the recess under the bridge-like fastening element is designed to be trough-shaped. In other embodiments, this recess is designed as a continuous groove up to the end of the side part. In the embodiments with such recesses, the clear height between the proximal surface of the side part and the inner side of the bridging portion is in relation to the deepest point of the groove-like or trough-like recess.

At that end of the groove-like recess facing in the direction of the central region, the groove bottom rises to the level of the proximal surface in certain embodiments. A retaining strap inserted in this direction is thus guided in the proximal direction and can thus be more easily gripped by the user.

In order to particularly effectively reduce the risk of a tilting load occurring in the event of unilateral tensioning of the cannula strap, the minimum distance of the pillar portions from the end of the respective side part in a preferred embodiment is at least 2 mm. Even better results can be achieved with certain embodiments in which the minimum distance is at least 4 mm. In particular embodiments, the minimum distance of the pillar portions from the end of the respective side part is at least 6 mm.

The geometry of the bridge-like fastening element is relatively variable. In particular, the present invention covers bridge-like fastening elements in which the two pillar portions of the respective fastening element form, with the bridging portion extending between the two pillar portions, in each case an angle in the range of 90° to 135°, or the two pillar portions of the respective fastening element, with the bridging portion extending between the two pillar portions, describe a circular arc. The invention also covers embodiments in which the upper face of the bridge-like fastening element is designed to be polygonal and the inner face facing in the direction of the eyelet opening is configured as a circular arc, and vice versa. Here, the term “polygonal” is to be understood to also include embodiments with corners that are rounded or chamfered in order to avoid sharp edges or corners.

In certain embodiments, the two pillar portions of the respective fastening element have a rectangular or a roundedly rectangular cross section. In alternative embodiments, the pillar portions have an oval or circular cross section.

In certain embodiments, the bridging portion extending between the two pillar portions has a rectangular or a roundedly rectangular cross section. In alternative embodiments, the bridging portion has an oval or circular cross section.

Overall, it holds true that rounded edges and corners offer advantages from the point of view of hygiene. More angular embodiment variants may in some cases offer advantages in terms of fastening and holding function, depending on the fastening technique used for the retaining strap.

To ensure that the end of the retaining strap can be easily gripped after it has been threaded through the eyelet, in certain embodiments a lifting element is provided on the proximal surface between the eyelet and the central region, which lifting element provides a lifting surface which, on the side toward the eyelet, ends substantially flush with the proximal surface and, in the direction of the central region, rises by 1 to 5 mm or 1 to 3 mm, as a result of which the end of a retaining strap guided through the eyelet from the direction of the side-part end toward the central region is slightly raised off the proximal surface of the side part, so that this end can be gripped more easily.

In certain embodiments, the two pillar portions of the respective fastening element stand at a right angle on the proximal surface of the shield. In alternative embodiments, the pillar portions are positioned at an angle that deviates by +/−45° from the right angle. In certain embodiments, the deviation from the right angle is either absolutely identical or at least quantitatively identical in the two pillar portions of a fastening element. In addition, the two pillar portions of a fastening element can also deviate with respect to the central longitudinal axis of the central region either in the radial direction or in the tangential direction.

The bridge-like fastening element provided according to the invention on the side parts provides an eyelet through which the retaining strap can be guided and to which the retaining strap can be fastened. However, since there are also users who would like to have the possibility of being able to thread the cannula strap alternatively through a conventional eyelet, depending on the given circumstances, certain embodiments of the present invention provide, in addition to the eyelet formed by the bridge-like fastening element, a conventional eyelet, the latter being provided in the shield surface in the region spanned by the bridge-like fastening element. At this location, the side parts thus have recesses which are continuous from the proximal surface to the distal surface and which, for example, may be designed to be rectangular, circular or oval or designed as an oblong hole. In the embodiments with such recesses, the clear height between the proximal surface of the side part and the inner side of the bridging portion is in relation to the plane of the proximal surface in the edge region of the recess.

If, in a first dimension on the plane of the proximal surface of the side part, the continuous recesses have a greater extent than in a second dimension, they may be oriented similarly to what has already been described for the bridge-like fastening elements. In the longitudinal direction of the recess, the latter can extend, for example, on a line which runs parallel to a projection of the central longitudinal axis of the side part on its proximal surface. Alternatively, the longitudinal direction of the recess can also lie on a line that intersects the projection of the central longitudinal axis of the side part on its proximal surface at an angle of 45 to 90°.

In a preferred embodiment, the continuous recesses through the cannula shield protrude in the direction of the end of the side part by about 1 to 3 mm beyond the region spanned by the bridge-like fastening element. This facilitates the threading of the cannula strap or else, if necessary, the suturing thereof to the patient's neck.

The line on which an elongate recess extends in its longitudinal direction on the proximal surface either lies on the line on which the pillar portions of the respective fastening element stand on the proximal surface, or it runs parallel thereto, or it intersects it on the radially outer side of the fastening element at an angle of 5 to 60°, preferably at an angle of 15 to 45°. In relation to the central transverse axis of the shield, the arrangement of the two continuous recesses of the shield can be mirror symmetrical or non-mirror symmetrical.

In certain embodiments, the side surface extending between the distal surface and the proximal surface of a side part runs over the width of the shield on an arc-shaped path at the end of the side part, such that the ends of the side parts are rounded. In some embodiments, the ends of the side part are also curved in an arc shape in the proximal direction so that, at the end of the side part, there is no edge that can come to rest on the patient's neck.

In order to generally avoid edges that may come to rest on the patient's neck, in certain embodiments of the invention the side surfaces extending between the distal surface and the proximal surface of a side part transition seamlessly, without forming an edge, into the distal surface and optionally also into the proximal surface of the respective side part.

The shield according to the invention is typically made of a plastic material. In certain embodiments, the shield is made of a soft, elastically deformable material, e.g. of soft PV, polyurethane, SEBS or another thermoplastic elastomer or of silicone. In alternative embodiments, the shield is made of a hard, dimensionally stable material, e.g. polystyrene, polycarbonate, ABS, polyamide or polyester. In particular embodiments, the shield is made partially of a hard, dimensionally stable material and partially of a soft, elastically deformable material.

In the embodiments in which the shield is made partially of a dimensionally stable material and partially of a soft, elastically deformable material, the distribution of the various materials is preferably as follows. The bridge-like fastening element and the region of the shield on which the bridge-like fastening element stands, and optionally at least in sections also the region which is provided around an optionally provided continuous recess in the side part of the shield, are preferably made of a hard, dimensionally stable material, while the remaining regions of the shield are made of a soft, elastically deformable material. In this way, the stability required for fastening the retaining strap is ensured in this region, while at the same time most of the surfaces lying on the patient's neck are made of a soft, elastically deformable material that is more comfortable for the patient.

In order to give the shield greater rigidity and stability, especially in the central region, in certain embodiments the regions of the two shield parts, which are made of hard material in the region of the fastening elements, are connected by a “stabilizing bridge” made of the same hard material, this stabilizing bridge extending over the central region of the shield.

Here, hard materials are understood to mean plastics with a Shore A hardness of ≥ 90, and soft plastics here are understood to be those with a Shore A hardness of 30 to 85. In the embodiments in which the shield is made of a uniform plastic material, the Shore A hardness of this material is preferably ≥40 in order to ensure a certain minimum stability of the entire shield.

The side parts of the shield are typically designed to be flat and preferably have a material thickness in the range of 1.5 to 4 mm. From the central region to the end of the side parts, the side parts can either have a continuously identical material thickness or a material thickness that decreases uniformly or in steps in the process. However, in certain embodiments of the invention, the thickness of the side parts of the shield may also vary in another way from the central region to the end of the respective side part. In the embodiments with side parts of continuously equal thickness, the distal surface of the side parts runs substantially parallel to the proximal surface of the respective side part.

In certain embodiments, the distal surface of the shield according to the invention has, in the central region, a proximally concavely curved portion in the longitudinal direction of the shield. In some embodiments, the distal surface of the shield according to the invention has, in the central region, a proximally concavely curved portion perpendicular to the longitudinal direction of the shield. In particular embodiments of the shield according to the invention, the distal surface has, in the central region, a proximally concavely curved configuration both in the longitudinal direction of the shield and in a direction perpendicular to the longitudinal direction of the shield.

The greatest distance of the distal surface from a tangential surface passing through the vertices, caused by the concave curvature of a portion of the central region, is between 4 mm and 20 mm. Preferably, the greatest distance is between 6 mm and 12 mm. In this way, it is readily ensured in particular that no pressure points are created on the patient's neck by shield sections lying on the latter.

To be able to optimally adapt the shield to the particular anatomy of the respective patient when applying it to the patient's neck, the shield in certain embodiments has, in the central region, a suspension device for cardanic mounting on the cannula tube or on a connector of a tracheostomy cannula.

In addition or alternatively, for the purpose of adaptation to the particular anatomy of the respective patient, in certain embodiments a separate flat cushion element made of a soft, elastically deformable material is provided bearing on the distal surface of the shield, wherein the outer circumference of the flat cushion element protrudes beyond the outer circumference of the distal surface of the shield by at least 1 mm on all sides.

The shield according to the invention is connected to the cannula or to the connector, and the cushion element can be plugged on beforehand or also retrofitted if necessary. Ideally, the shield thus fulfills its function with the properties described further above, even without the variant described here with a cushion element. The cushion element serves either to further improve the properties of the shield according to the invention or to permit individualization to the special requirements of a particular patient.

The cushion element can be provided for a standard shield according to the invention either individually or in the form of a kind of adapter set in different shapes and sizes, in order then to be able to use the selected cushion element to adapt the shield in situ to the particular anatomy and the individual needs of the respective patient.

In some embodiments, a conical connector piece, which can be inserted into a patient's stoma to provide a better seal, is located on the distal surface of the cushion element, in the region which comes to lie on the central region of the shield. The outer diameter, height and slope can vary depending on the size of the respective patient's stoma.

The material of the cushion element preferably has a thickness of at least 0.2 mm in order to achieve the best possible cushioning effect. However, with the aid of the cushion element, it can also be ensured that a cannula that is slightly too long for a certain patient is inserted less deeply into the trachea. Therefore, in some embodiments, the material of the cushion element has a thickness of up to 12 mm. In certain embodiments, the thickness is in the range of 0.2 mm to 8 mm or in the range of 0.2 mm to 4 mm.

Examples of soft elastomeric materials that can be used for the cushion element have already been mentioned above in connection with soft components of the shield.

In particular embodiments, the material of the cushion element forms an elastic hollow body, as a result of which the thickness of the cushion element can be varied by filling the hollow body with different volumes of a filling fluid. In embodiments with a conical connector piece, the latter may also be provided with a hollow body, as a result of which expansion of the connector piece, by filling the hollow body with different volumes of a filling fluid, is optimally adapted to the size of the stoma. The more fluid that is added, the larger the diameter of the conical seal.

In some embodiments, the shield and the cushion element are adhesively bonded to each other. In alternative embodiments, the cushion element is held on the shield by a form-fit or force-fit connection, e.g. a snap closure. In certain embodiments, the shield and the cushion element are connected to each other by a two-component injection molding process.

A particular embodiment of this variant is characterized in that the shield is made of a hard material, and continuous recesses from the proximal surface to the distal surface are provided in the side parts of the shield, which recesses extend at least over the region that extends next to the region spanned by the bridge-like fastening elements. The separate flat cushion element bearing on the distal surface of the shield is made of a soft, elastically deformable material, wherein the outer circumference of the flat cushion element protrudes beyond the outer circumference of the proximal surface of the shield by at least 1 mm on all sides.

In the embodiment described here, the separate cushion element has two eyelets for fastening a retaining strap, wherein the eyelets are each formed by a bridge-like fastening element which has two pillar portions arranged on the proximal surface of the cushion element and a bridging portion extending between the two pillar portions. The two bridge-like fastening elements of the cushion element are provided here in such a way that they protrude through the continuous recesses arranged in the side parts of the shield and are thereby arranged next to the bridge-like fastening elements of the shield such that the eyelets of the cushion element come to be substantially congruent with the eyelets of the shield. Preferably, in the assembled state, the pillars of the fastening elements of the cushion element are also located at a distance from the end of the respective side part of the shield.

Since the fastening elements of the shield and cushion element are arranged next to each other in this embodiment, the retaining strap can be guided through in each case both fastening elements. In this way, the components can no longer detach from each other. This can therefore prevent the cushion element from accidentally slipping into the trachea.

The present invention relates to a shield for mounting on a cannula tube of a tracheostomy cannula or on an annular connector arranged proximally on the cannula tube. However, the present invention also covers tracheostomy cannulas for insertion into a tracheostoma, having a cannula tube and a shield of the type according to the invention described above.

For the purposes of the original disclosure, it is pointed out that all the features which a person skilled in the art can gather from the present description, the drawings and the claims, even if they have been described specifically only in connection with certain further features, can be combined both individually and in any desired combinations with other features or groups of features disclosed herein, provided that this has not been expressly ruled out or such combinations are rendered impossible or pointless for technical reasons. Here, purely for the sake of brevity and readability of the description, the comprehensive and explicit presentation of all conceivable combinations of features is omitted.

It will furthermore be noted that it is self-evident to a person skilled in the art that the following exemplary embodiments serve merely to provide some examples of the possible embodiments of the present invention represented as exemplary embodiments. A person skilled in the art will therefore readily appreciate that, in addition, all other embodiments having the features or combinations of features according to the invention specified in the claims also fall within the scope of protection of the invention. Here, purely for the sake of brevity and readability of the description, the comprehensive and explicit presentation of all conceivable embodiments is omitted.

Some of the abovementioned features or combinations of features are illustrated in the appended figures in the form of exemplary embodiments, without the present invention being in any way intended to be limited to individual examples of the embodiments shown here.

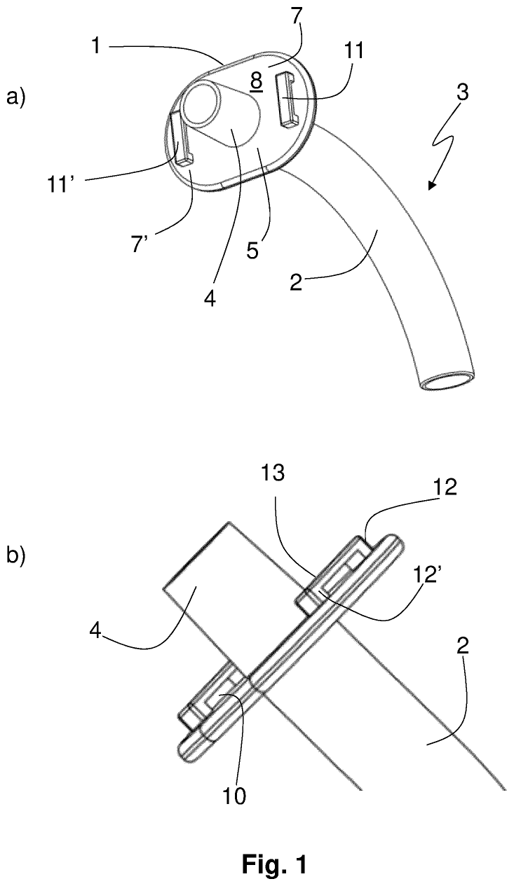

FIG. 1 shows an embodiment of a shield according to the invention, mounted on a cannula tube of a tracheostomy cannula and having a polygonal bridge-like fastening element.

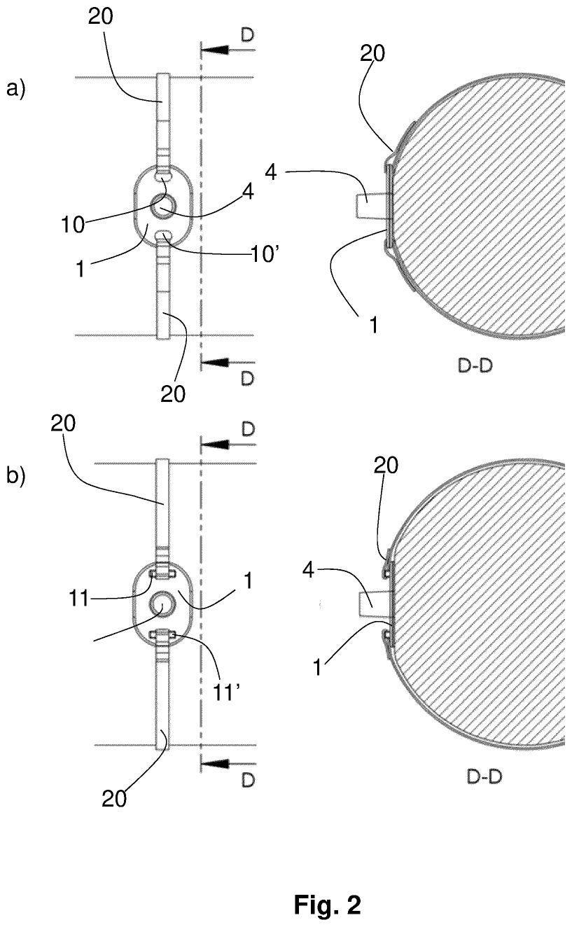

FIG. 2 shows the comparison between a conventional shield from the prior art, which has fastening eyelets provided in the form of continuous recesses in the shield surface (FIG. 2a), having a shield according to the invention, in which the eyelets are provided by a bridge-like fastening element arranged on the proximal surface of the shield (FIG. 2b).

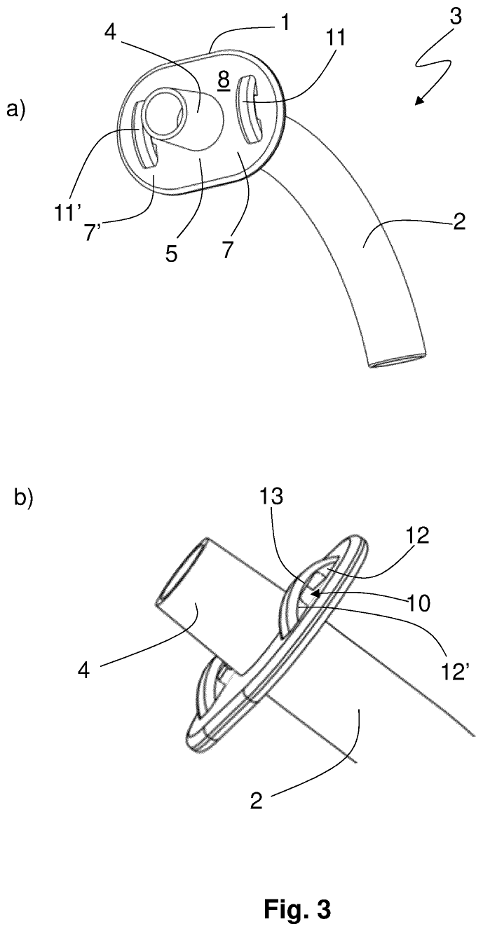

FIG. 3 shows an embodiment of a shield according to the invention, mounted on a cannula tube of a tracheostomy cannula and having a rounded bridge-like fastening element.

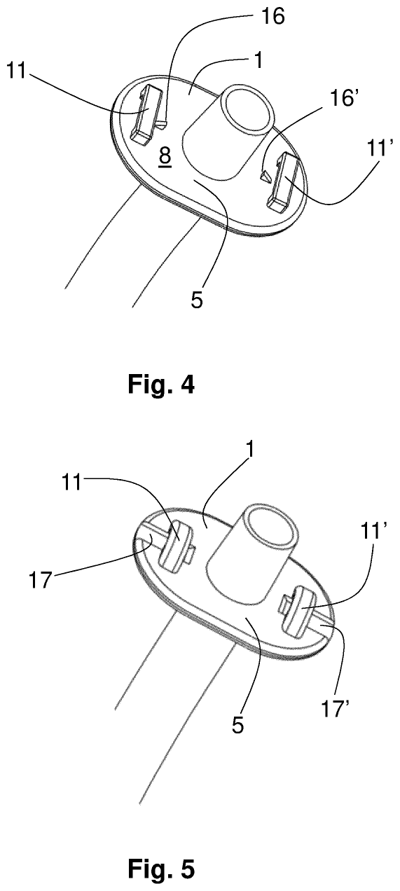

FIG. 4 shows an embodiment of a shield according to the invention with lifting elements for the retaining strap.

FIG. 5 shows an embodiment of a shield according to the invention with groove-shaped recesses in the shield surface, through which the retaining strap can be guided.

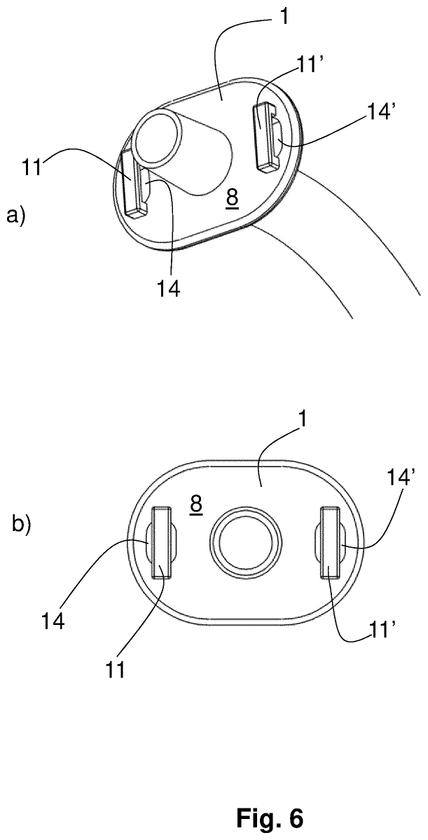

FIG. 6 shows an embodiment of a shield according to the invention, in which continuous recesses are provided in the shield surface, under the bridge-like fastening elements, through which recesses the retaining strap can be guided.

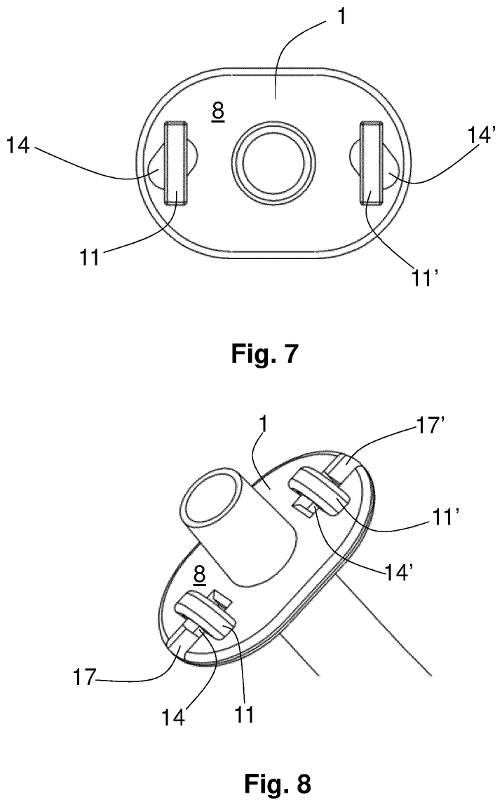

FIG. 7 shows an alternative embodiment of a shield according to the invention in which, under the bridge-like fastening elements, continuous recesses are provided which are oriented slightly differently compared to the embodiment of FIG. 6.

FIG. 8 shows an embodiment of a shield according to the invention having a combination of groove-shaped recesses and continuous recesses in the region of the bridge-like fastening elements.

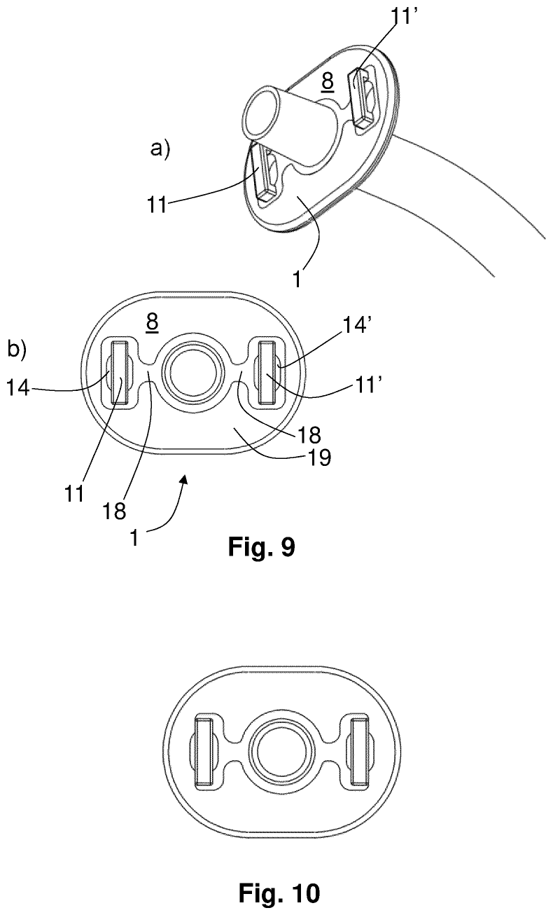

FIG. 9 shows an embodiment of a shield according to the invention, in which the material of the shield is formed in some sections from a harder plastic material and in some sections from a softer plastic material.

FIG. 10 shows in comparison to FIG. 9 an alternative embodiment of a shield according to the invention with regions of differently hard plastic materials.

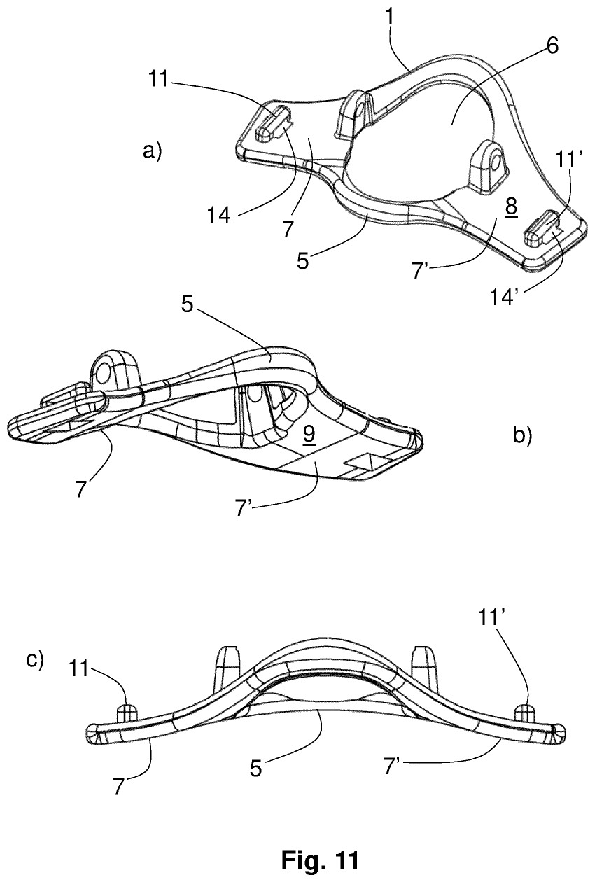

FIG. 11 shows an embodiment of a shield according to the invention with curved shield surfaces.

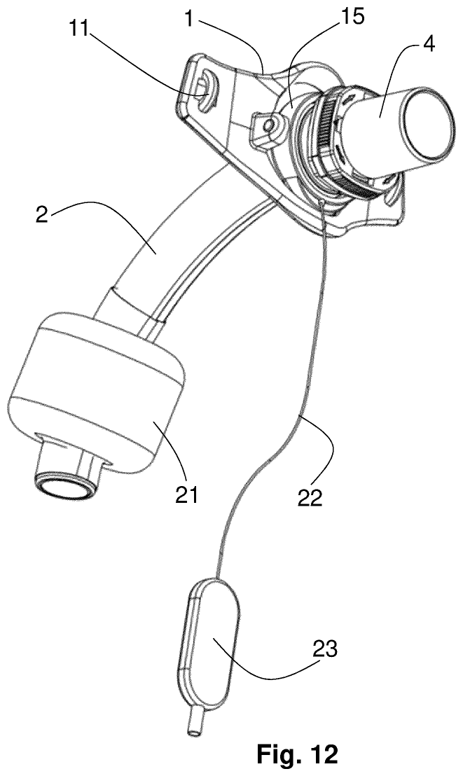

FIG. 12 shows a tracheostomy cannula with a cardanically mounted shield according to the invention.

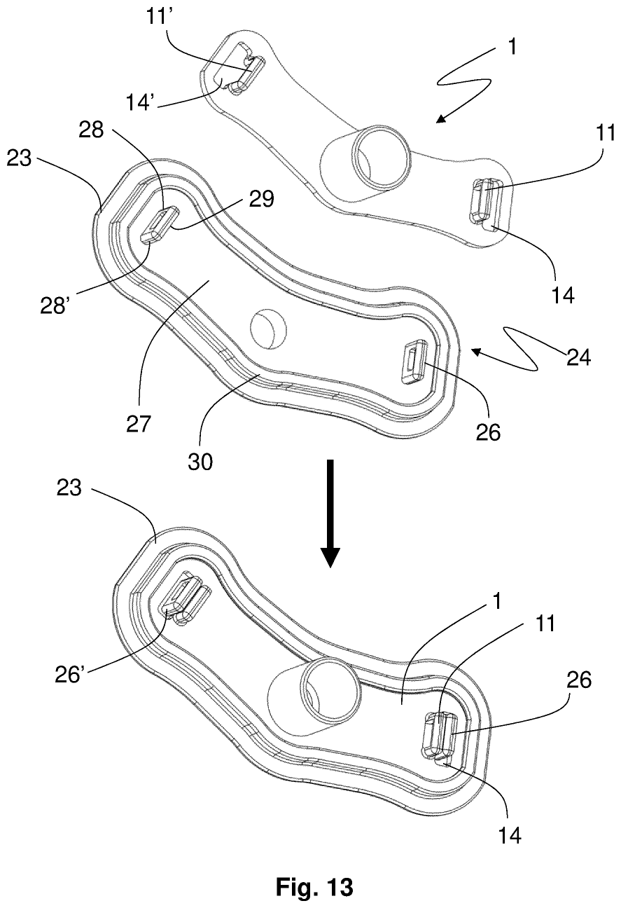

FIG. 13 shows an embodiment of a shield according to the invention with a cushion element, wherein shield and cushion element are shown once separately (top) and are shown once in the assembled state (bottom).

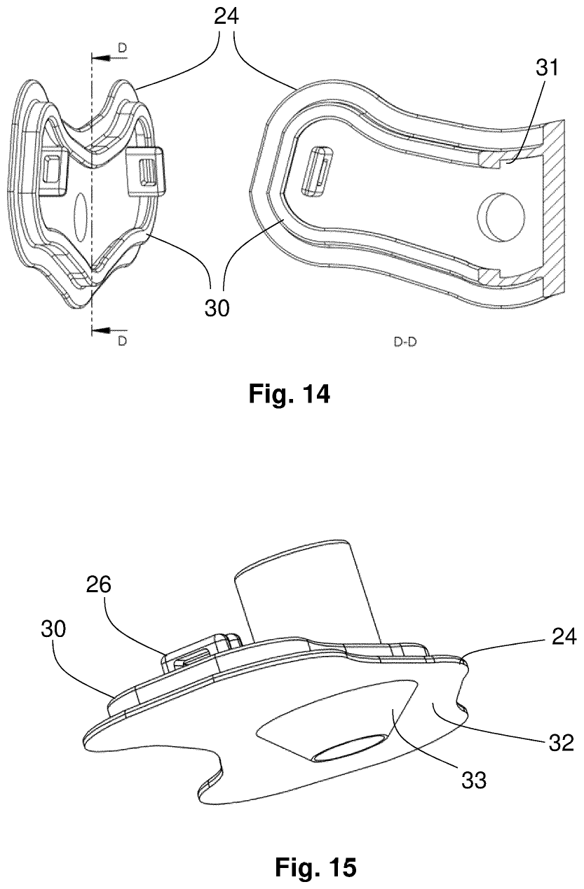

FIG. 14 shows a special variant of the cushion element of a shield according to the invention with a snap closure provided on the shield, seen once in a plan view from the side (on the left) and once in a detail of the cushion element in the sectioned state.

FIG. 15 shows an embodiment of a shield according to the invention with a cushion element which has a conical connection piece on the underside.

FIG. 1a) is a perspective plan view of a tracheostomy cannula 3 with a shield 1 according to the invention arranged thereon. Located at the proximal end of the cannula tube 2 is a connector 4, which is guided through a corresponding recess in the central region 5 of the shield 1. This view shows the proximal surface 8 of the shield 1 extending over the central region 5 and the two side parts 7, 7′, the latter adjoining two opposite sides of the central region 5 and extending radially from there to the respective end of the side part 7, 7′. In the end region of the side parts 7, 7′, a respective bridge-like fastening element 11, 11′ is arranged which has two pillar portions 12, 12′ on the proximal surface 8 of the shield and a bridging portion 13 extending between the two pillar portions 12, 12′ (cf. FIG. 1b). The pillar portions 12, 12′ are arranged at a distance from the end of the respective side part, so that the bridge-like fastening elements 11, 11′ are arranged overall at a distance from the respective end of the side part 7, 7′ so as to form in each case an eyelet 10, 10′ there. This eyelet 10, 10′ extends across the region extending across the proximal surface 8 of the shield 1 over the region spanned by the bridge-like fastening element 11, 11′.

In the embodiment shown here, the bridge-like fastening element 11, 11′ is designed to be polygonal, wherein the edges and corners of the bridge-like fastening element 11, 11′ are designed to be rounded in order to keep the potential risk of injury at these corners and edges as low as possible.

In FIG. 1b), the proximal region of the tracheostomy cannula 3 shown in FIG. 1a) is shown greatly enlarged and in a different perspective in order to be able to illustrate the pillar portions 12, 12′ and the bridging portion 13 of the fastening elements 11, 11′ more clearly. In addition, the eyelet 10, 10′ formed by the bridge-like element 11, 11′ in interaction with the proximal surface 8 of the shield 1 can also be better seen in this illustration.

FIG. 2a) shows a conventional shield 1 as known from the prior art. In this conventional shield 1, the eyelets 10, 10′ for the retaining strap 20 are provided in the form of continuous recesses in the shield 1. The left-hand illustration in FIG. 2a) shows a plan view of the conventional shield 1 with a retaining strap 20 arranged thereon. Here, the shield 1 is shown in the state fixed to a patient's neck with the aid of the retaining strap 20.

The right-hand illustration in FIG. 2a) shows the section along the line D-D, in order to show that, in this conventional solution, the retaining strap 20 is partially guided under the shield 1, i.e. between the shield 1 and the patient's neck (shaded region).

FIG. 2b) shows a shield according to the invention with fastening elements 11, 11′ and a retaining strap 20 arranged thereon likewise in the state applied to the patient's neck. The left-hand illustration in FIG. 2b) shows a plan view from above of the shield 1, while the illustration on the right shows a section along the line D-D. It will be clearly seen from this illustration that, in the case of a shield according to the present invention, the retaining strap 20 is not guided between the shield and the patient's neck, and therefore the undesired frictional effects cannot arise here either.

FIG. 3a) shows an alternative embodiment of a shield according to the invention which is mounted on the cannula tube 2 of a tracheostomy cannula 3 in the region of the connector 4. In the embodiment shown here, the bridge-like fastening elements 11, 11′ arranged in each case at a distance from the ends of the side parts 7, 7′ are designed to be rounded, wherein the pillar portions 12, 12′ transition seamlessly into the bridging portion 13.

In FIG. 3b), the proximal end of the embodiment of the invention shown in FIG. 3a) is shown greatly enlarged and in a different perspective, in which the eyelets 10, 10′ which are formed by the fact that the bridge-like fastening elements 11, 11′ are arranged on the proximal surface 8 of the shield 1 according to the invention are formed.

FIG. 4 shows an embodiment of a shield according to the invention, in which lifting elements 16, 16′ are provided on the proximal surface 8 between the bridge-like fastening elements 11, 11′ and the central region 5. These lifting elements 16, 16′ have a lifting surface which rises in the direction of the central region 5 and which, on the side toward the eyelet, ends substantially flush with the proximal surface 8 of the shield 1. By means of the end of the lifting surface rising in the direction of the central region, the end of a retaining strap (not shown) guided through the eyelet from the direction of the side part end in the direction of the central region 5 is lifted slightly off the proximal surface of the side part, so that this end can be easily gripped.

FIG. 5 shows an embodiment of a shield 1 according to the invention, in which the bridge-like fastening elements 11, 11′ are designed to be rounded, wherein the total height of the bridge-like fastening elements 11, 11′ can be kept relatively low here, since groove-like recesses are provided under the bridge-like fastening elements 11, 11′ and extend continuously to the end of the side part. In this way, an eyelet with a sufficiently large clear height is achieved, such that the retaining strap can be easily guided through. In the direction of the central region 5, the groove bottom in the embodiment shown here rises to the level of the proximal surface 8. A retaining strap inserted in this direction is thus guided in the proximal direction and can thus be easily gripped by the user.

FIG. 6 shows an embodiment of a shield 1 according to the invention in which, under the bridge-like fastening elements 11, 11′, continuous recesses 14, 14′ are provided from the proximal surface to the distal surface of the shield. In the embodiment shown here, the continuous recesses 14, 14′ are provided in the form of oblong holes which extend from the inner side of one pillar portion of a bridge-like fastening element 11, 11′ to the inner side of the other pillar portion of the same bridge-like fastening element 11, 11′. These continuous recesses 14, 14′ offer the possibility of threading the cannula strap alternatively through the continuous recesses, depending on the given circumstances, if this is desired by the user. FIG. 6a) shows this particular embodiment in a perspective view. In FIG. 6b), the same embodiment is again shown in a plan view from above of the proximal surface 8 of the shield 1.

FIG. 7 shows an alternative embodiment of a shield 1 according to the invention with continuous recesses 14, 14′ arranged under the bridge-like fastening elements 11, 11′, wherein the continuous recess is elongate, and wherein the longitudinal axis of the recess, in contrast to the embodiment shown in FIG. 6, lies on a line which at an angle of approximately 45° intersects the line on which the pillar portions of the fastening element stand on the proximal surface.

FIG. 8 shows an alternative embodiment of the shield 1 according to the invention with continuous recesses 14, 14′ arranged under the bridge-like fastening elements 11, 11′, wherein in this embodiment groove-like recesses 17, 17′ are additionally provided which, on the continuous recesses 14, 14′, extend continuously in the direction of the lateral ends of the shield 1.

FIGS. 9 and 10 show alternative embodiments of the shield according to the invention. In the embodiments shown here, continuous recesses 14, 14′ are provided in addition to the bridge-like fastening elements 11, 11′. A particular aspect of the embodiments shown here is that the shield material in some regions of the shield 1 consists of a harder plastic material and in other regions consists of a softer plastic material. The regions 18 with harder plastic material extend at least partially or completely around the bridge-like fastening elements 11, 11′, the continuous recesses 14, 14′, and the continuous recess which is arranged in the central region of the shield and through which a cannula or a connector is guided. This serves to provide particularly high stability in these regions, since these regions are particularly important for fixing the retaining strap and for stabilizing the cannula. By contrast, the other regions 19, which are made of a softer plastic material, ensure better wearing comfort and less mechanical stress on the patient's neck.

FIG. 11 shows various views of a particular embodiment of a shield 1 according to the invention, in which both the proximal surface 8 of the shield and the distal surface 9 of the shield are designed to be curved in a particular manner. In this case, the central region 5 is curved in the proximal direction such that it does not come to rest on the patient's neck when the shield 1 is fastened to the patient's neck with a retaining strap. This avoids unnecessary rubbing on the patient's neck in this region.

FIG. 12 shows an embodiment of a shield 1 according to the invention, which is arranged on the cannula 2 via a cardanic mounting 15. The additional mobility of the shield 1, achieved through the cardanic mounting 15, enables optimal individual adaptation to the given neck anatomy of the respective patient.

FIG. 13 shows an embodiment of a shield according to the invention with a cushion element. In the upper part, the shield 1 and the cushion element 24 are shown separately, i.e. in the non-assembled state. Below the arrow shown in the figure, the assembled state is then shown in which the shield 1 is arranged on the cushion element 24.

As can be seen in particular from the upper illustration, the fastening elements 11, 11′ are arranged on the proximal surface of the shield 1 at a distance from the end of the side parts. In addition, the continuous recesses 14, 14′ are located there. Furthermore, the cushion element 24 with the fastening elements 26, 26′ can be clearly seen in the upper illustration. As in the case of the fastening elements 11, 11′ arranged on the shield 1, the fastening elements 26, 26′ on the cushion element 24 also consist of pillar portions 28, 28′ and an intermediate bridging portion 29, such that a bridge-like fastening element is obtained which forms an eyelet through which a retaining strap can be guided.

The cushion element 24 is designed to be substantially flat, apart from the fastening elements 26, 26′ standing on the proximal surface 27. However, in the embodiment shown here, a completely circumferential annular bead 30 is provided, which is provided for holding the shield 1 in the cushion element 24. According to the invention, the substantially flat cushion element is made of a soft, elastically deformable material, and the geometric shape is chosen such that the outer circumference of the flat cushion element 24 protrudes beyond the outer circumference of the distal surface of the shield 1 by at least 1 mm on all sides.

In the assembled illustration below the case in arrow 13, it can be clearly seen that the fastening elements 26, 26′ of the cushion element 24 are arranged in such a way that they protrude through the continuous recesses 14, 14′ arranged in the side parts of the shield 1 and are thereby arranged next to the bridge-like fastening elements 11, 11′ of the shield 1 such that the eyelets of the cushion element 24 come to be substantially congruent with the eyelets of the shield 1. A retaining strap guided through the eyelets of the fastening elements 11, 11′ of the shield 1 can thus equally also be guided through the eyelets formed by the fastening elements 26, 26′ of the cushion element 24. The shield 1 and the cushion element 24 are thus held in position by the same retaining strap, both in relation to each other and to the patient's neck.

In FIG. 14, the cushion element 24 of the embodiment of the shield 1 according to the invention shown in FIG. 13 is shown with the cushion element 24 once in a plan view from the side (left-hand side of FIG. 14). The right-hand side of FIG. 14 shows a sectioned detail view of the cushion element 24 along the section plane D-D, as indicated in the illustration on the left-hand side of FIG. 14. In particular, the section detail shows clearly the annular bead 30, which is provided on the proximal surface of the cushion element 24. Also recognizable here is the undercut 31, which is designed such that a shield mounted on the cushion 24 can slip into the undercut 31 of the annular bead 30, since the annular bead 30, like the entire cushion 24, is also made of a soft, elastic material. The shield is thus held in a form-fitting manner on the cushion 24 (see lower illustration in FIG. 13).

FIG. 15 shows a particular embodiment of a cushion element 24 for the shield according to the invention. In the embodiment shown here, a conical connecting piece 33 is located on the distal surface 32 of the cushion element 24 and, particularly in patients with a relatively large stoma, can serve to provide a better seal, so that as little speaking air as possible can escape at this point. Depending on the size of the respective patient's stoma, a cushion element 24 can be used which has a conical portion of a correspondingly suitable outer diameter, suitable height and suitable slope.

### LIST OF REFERENCE SIGNS

1 shield
2 cannula tube
3 tracheostomy cannula
4 connector
5 central region
6 continuous recess
7, 7′ side part
8 proximal surface
9 distal surface
10, 10′ eyelet
11, 11′ fastening element
12, 12′ pillar portions
13 bridging portion
14, 14′ continuous recess
15 cardanic mounting
16, 16′ lifting element
17, 17′ groove-shaped recess
18 region made of hard plastic material
19 region made of soft plastic material
20 retaining strap
21 cuff
22 cuff supply line
23 filling balloon
24 cushion element
25, 25′ eyelet of the cushion element
26, 26′ fastening element of the cushion element
27 proximal surface of the cushion element
28, 28′ pillar portions
29 bridging portion
30 annular bead
31 undercut
32 distal surface of the cushion element
33 conical portion

1. A shield (1) for mounting on a cannula tube (2) of a tracheostomy cannula (3) or on an annular connector (4) arranged proximally on the cannula tube (2), wherein the shield (1) has
a central region (5) comprising a continuous recess (6) through which the cannula tube (2) and/or the connector (4) is guided, and
two side parts (7, 7′) which adjoin two opposite sides of the central region (5) and extend radially from there to the respective end of the side part (7, 7′),
wherein the shield (1) has a proximal side with a proximal surface (8), which faces away from the patient's neck during use, and has a distal side with a distal surface (9), which faces toward the patient's neck during use, wherein the two side parts each have an eyelet (10, 10′) for fastening a retaining strap,
characterized in that
the respective eyelet (10, 10′) is formed by a bridge-like fastening element (11, 11′), which has two pillar portions (12, 12′) arranged on the proximal surface (8) of the shield (1)
and a bridging portion (13) which extends between the two pillar portions (12, 12′), wherein both pillar portions (12, 12′) are arranged at a distance from the end of the respective side part (7, 7′).

2. The shield as claimed in claim 1, characterized in that the distance between the inner sides of the pillar portions of a bridge-like fastening element is in the range of 3 to 12 mm, and/or the clear height between the proximal surface of the shield and the inner side of the bridging portion is in the range of 2 to 5 mm.

3. The shield as claimed in either of claims 1 and 2, characterized in that the distance of the pillar portions from the end of the respective side part is at least 4 mm, more preferably at least 6 mm.

4. The shield as claimed in any one of the preceding claims, characterized in that the two pillar portions of the respective fastening element and the bridging portion extending between the two pillar portions have a rectangular, a roundedly rectangular, an oval or circular cross section.

5. The shield as claimed in any one of the preceding claims, characterized in that the two pillar portions of the respective fastening element form, with the bridging portion extending between the two pillar portions, in each case an angle in the range of 90° to 135°, or the two pillar portions of the respective fastening element, together with the bridging portion extending between the two pillar portions, describe a circular arc.

6. The shield as claimed in any one of the preceding claims, characterized in that the two pillar portions of the respective fastening element stand at a right angle on the proximal surface, or at an angle that deviates by +/−45° from the right angle.

7. The shield as claimed in any one of the preceding claims, characterized in that both side parts, in the region spanned by the fastening element, have a recess (14, 14′) which is continuous from the proximal surface to the distal surface and which, in the direction of the end of the side part, optionally protrudes by about 1 to 3 mm beyond the region that is spanned by the bridge-like fastening element.

8. The shield as claimed in any one of the preceding claims, characterized in that the distal surface of the side parts runs substantially parallel to the proximal surface of the respective side part.

9. The shield as claimed in any one of the preceding claims, characterized in that the side surface which extends between the distal surface and the proximal surface of a side part runs, at the end of the side part, over the width of the shield on an arc-shaped path and/or, without forming an edge, transitions seamlessly into the distal surface and optionally also into the proximal surface of the respective side part.

10. The shield as claimed in any one of the preceding claims, characterized in that the shield is made of a soft, elastically deformable material or of a hard, dimensionally stable material or partly of a soft, elastically deformable material and partly of a hard, dimensionally stable material.

11. The shield as claimed in any one of the preceding claims, characterized in that the side parts of the shield are designed to be flat and preferably have a material thickness in the range of 1.5 mm to 4 mm.

12. The shield as claimed in any one of the preceding claims, characterized in that the distal surface in the central region has a proximally concavely curved portion in the longitudinal direction of the shield and/or a proximally concavely curved portion perpendicular to the longitudinal direction of the shield.

13. The shield as claimed in any one of the preceding claims, characterized in that the shield has at least one of the following:
in the central region, a suspension device (15) for cardanic mounting on the cannula tube or on a connector of a tracheostomy cannula,
on the proximal surface between the eyelet and the central region, a lifting element (16, 16′) having a lifting surface which, on the side toward the eyelet, ends substantially flush with the proximal surface and, in the direction of the central region, rises by 1 to 5 mm or 1 to 3 mm,
in the proximal surface, in each case a trough-shaped or groove-like recess (17, 17′) in and optionally beyond the region spanned by the bridge-like fastening elements,
in the side parts, recesses (14, 14′) which are continuous from the proximal surface to the distal surface and which extend at least over the region spanned by the bridge-like fastening elements, or which extend at least over the region extending adjacent to the region spanned by the bridge-like fastening elements,
a separate flat cushion element (24) made of a soft, elastically deformable material and bearing on the distal surface of the shield, wherein the outer circumference of the flat cushion element protrudes beyond the outer circumference of the distal surface of the shield by at least 1 mm on all sides.

14. The shield as claimed in any one of the preceding claims, characterized in that, in the side parts, recesses (14, 14′) are provided which are continuous from the proximal surface to the distal surface and which extend at least over the region extending adjacent to the region spanned by the bridge-like fastening elements, wherein the shield has a separate flat cushion element (24) made of a soft, elastically deformable material and bearing on the distal surface of the shield, wherein the outer circumference of the flat cushion element protrudes beyond the outer circumference of the distal surface of the shield by at least 1 mm on all sides, wherein the separate cushion element (24) has two eyelets (25, 25′) for fastening a retaining strap, wherein the respective eyelet (25, 25′) is formed by a bridge-like fastening element (26, 26′) which has two pillar portions (28, 28′) arranged on the proximal surface (27) of the cushion element (24) and a bridging portion (29) extending between the two pillar portions (28, 28′), wherein the two bridge-like fastening elements (26, 26′) of the cushion element (24) are provided such that they protrude through continuous recesses (14, 14′) arranged in the side parts of the shield (1) and are thus arranged next to the bridge-like fastening elements (11, 11′) of the shield such that the eyelets (25, 25′) of the cushion element (24) come to be substantially congruent with the eyelets (10, 10′) of the shield (1).

15. A tracheostomy cannula for insertion into a tracheostoma, with a cannula tube and a shield as claimed in any one of claims 1 to 14.
